(19) United States
(12) Patent Application Publication
(10) Pub. No.: US 2013/0251738 A1
(43) Pub. Date: Sep. 26, 2013

(54) VACCINES

(71) Applicants: Phillip C. Humphryes, Addlestone Surrey (GB); Nicholas G. Coldham, Addlestone Surrey (GB)

(72) Inventors: Phillip C. Humphryes, Addlestone Surrey (GB); Nicholas G. Coldham, Addlestone Surrey (GB)

(73) Assignee: The Secretary of State for Environment, Food & Rural Affairs, Worcester (GB)

(21) Appl. No.: 13/822,458

(22) PCT Filed: Sep. 13, 2011
(86) PCT No.: PCT/GB2011/051711

§ 371 Date: May 20, 2013

Related U.S. Application Data
(60) Provisional application No. 61/414,096, filed on Nov. 16, 2010.

(30) Foreign Application Priority Data
Sep. 13, 2010 (GB) 1015223.9

(51) Int. Cl. C07K 14/20; G01N 33/15; G01N 33/68; C12Q 1/06; G06F 19/18

(52) U.S. Cl. CPC C07K 14/20; C12Q 1/06; G06F 19/18; G01N 33/6878; G01N 33/15
USPC 424/190.1; 435/39; 530/350; 536/23.7; 435/7.1; 73/61.52; 250/282

(57) ABSTRACT

There is provided a method of identifying a protective Leptospira vaccine composition, comprising determining that the composition contains a protective concentration of a Lipl32 epitope polypeptide, for example, of a polypeptide comprising SEQ ID NO:13 or an antigenic variant or portion thereof. There is also provided a vaccine composition comprising a protective concentration of at least one Lipl32 epitope polypeptide having up to 250 amino acids and comprising SEQ ID NO:13 or an antigenic variant or portion thereof. There is further provided a method of identifying an immunogenic element in a vaccine composition comprising submitting the composition to a two-dimensional liquid chromatography mass spectrometry (2D LC/MS) process.

### FIELD OF INVENTION

The invention relates to methods for identifying protective vaccine compositions, as well as to vaccine compositions for use to protect an animal against infection by a Leptospira bacterium.

### BACKGROUND

Prevalent worldwide, Leptospira interrogans is a water borne zoonotic, responsible for leptospirosis, capable of infecting virtually any mammal it comes into contact with [1]. Primarily contracted through exposure with infected urine, symptoms can be diverse, ranging from fever to hepatic failure, but often result in death if left untreated [2]. Other symptoms, such as loss of milk production and miscarriage, are prevalent in cattle [3] making leptospirosis particularly damaging to the farming community. Vaccination is widely used to protect against infection, but unfortunately the vaccines currently available are serovar specific [4] and require regular boosters to maintain immunity [5]. Large volumes of vaccine are, therefore, produced every year, to keep the domestic animal population protected, with new variants under constant development.

Potency testing of Leptospira vaccines is required by law prior to new batches being released onto the market. The current test, as laid out by the European monograph [6], requires that, for each serovar for which the vaccine provides protection, ten hamsters be infected with a virulent strain of that serovar. Five of these receive the vaccine prior to infection but the other five, being the control group, do not. The end point of this test is death and euthanasia is not typically employed, to avoid skewing the results. Additionally, for reasons not fully understood, Leptospira has a predisposition to lose virulence during in vitro culture [7] and it is therefore routine practice to maintain the strains in vivo through continual hamster passage [8].

The use of such a large number of animals is ethically and financially undesirable and so an in vitro replacement for the potency test has been sought for some time. Previous attempts have failed due to their reliance on antibodies for characterisation of potency which, being of biological origin, are prone to attenuation. As the majority of this research was unsuccessful, there is a paucity of information in the literature as to the exact approaches employed. However, it is recorded [9] that both the microscopic agglutination test (MAT) and monoclonal antibody based ELISAs were utilised. Further, it was clearly established that protein and LPS [10], derived from Leptospira, were capable of eliciting an immune response.

Prior studies [21, 22] have successfully determined the proteome of Leptospira interrogans. The proteins Lipl32 (Hap-1), Lipl41, Lipl45 and OmpL1 have been identified as possible vaccine candidates [23, 16, 24] and, in some cases, trialled in vivo [20, 14]. However the exact mechanism by which Leptospira vaccines work remains poorly understood and to date no reliable method exists for their analysis or quantitation.

### SUMMARY OF INVENTION

According to a first aspect of the invention, there is provided a method of identifying a protective Leptospira vaccine composition, comprising determining that the composition contains a protective concentration (or abundance) of a Lipl32 epitope polypeptide.

For example, this may be determined by exposing the vaccine to a proteolytic enzyme (for example, trypsin or Proteinase K), processing the sample using a mass spectrometry method such as 2D-LC/MS, 1D-LC or gel-MS (for example), determining the Normalised Spectrum Abundance Factor (NSAF, for example determined as described herein) and determining that this is greater than the NSAF obtained from a known non-protective vaccine composition.

The term “protective Leptospira vaccine composition” indicates that the vaccine composition, when administered to an animal, is effective to protect the animal from infection by a bacterium of the genus Leptospira after exposure of the animal to the bacterium. Such protection can be verified by standard animal models such as are described by Marbehant in European Pharmacopeia Forum. 1999 p. 11-16. The bacterium may be, for example, L. interrogans, L. kirschneri, L. noguchii, L. alexanderi, L. weilii, L. genomospecies 1, L. borgpetersenii, L. santarosai, L. kmetyi, L. canicola or L. icterohaemorragiae. The vaccine composition may be protective against infection against any one or more of these species. The animal may be, for example, a cow, dog, horse, sheep, pig, rodent or human being.

The vaccine composition may comprise an attenuated Leptospira species selected, for example, from those listed above.

A non-protective vaccine composition, therefore, is one which does not fulfil the definition of a protective composition given above.

The method may comprise determining that the composition comprises a polypeptide comprising SEQ ID NO:13 or an antigenic variant or portion thereof. For example, the method may comprise exposing the composition to a proteolytic enzyme (such as trypsin) and subsequently detecting the presence of a protective concentration or abundance of SEQ ID NO:13 or an antigenic variant or portion thereof.

A protective concentration or abundance is a concentration or abundance correlated with vaccine compositions which are known to be protective, as has been shown by the inventors herein. For example, a protective concentration may be at least 0.25 fmol SEQ ID NO:13 polypeptide per μg of vaccine, for example at least 0.5 fmol SEQ ID NO:13 polypeptide per μg of vaccine. The vaccine may first have been exposed to a proteolytic enzyme (such as trypsin or Proteinase K), generating a pool of tryptic peptides in which SEQ ID NO:13 may be included.

The term “Lipl32 epitope polypeptide”, as used throughout this specification, indicates a peptide which contains one or more (or all) epitopes of Lipl32 protein and, furthermore, comprises SEQ ID NO:13 or an antigenic variant or portion thereof. Therefore, a Lipl32 epitope polypeptide may be a polypeptide comprising Lipl32 full length polypeptide (for example, SEQ ID NO:1) or a fragment thereof having up to 250 amino acids which contains at least one Lipl32 epitope. The Lipl32 epitope polypeptide may be Lipl32 (SEQ ID NO:1) or a functional fragment thereof, such as SEQ ID NO:13.

SEQ ID NO:1 is the sequence of Lipl32 from L. canicola and the skilled person will readily be able to identify the equivalent sequence in other species, for example by use of sequence identity databases. All such Lipl32 sequences and functional fragments thereof are encompassed by the present invention.

An “antigenic variant or portion” of SEQ ID NO:13, as referred to throughout this specification, is any sequence having equivalent antigenic properties as SEQ ID NO:13. For example, this would include a sequence which is capable of binding with an antibody which is itself capable of binding with the polypeptide having amino acid sequence SEQ ID NO:13.

Advantageously, the use of the method according to the invention enables the user to verify whether a new vaccine composition will be potent, i.e., whether administration of the vaccine to an animal will protect the animal from infection, without the need for animal testing verification such as is currently used. The user simply needs to determine the concentration (i.e., abundance) of the Lipl32 epitope polypeptide within the composition so as to confirm whether the composition is a potent vaccine, i.e., will provide protection from infection to an animal to which it is administered. When the Lipl32 epitope polypeptide is present at a protective concentration or abundance, the Normalised Spectrum Abundance Factor (NSAF, determined after exposure of the vaccine to a tryptic peptide and processing by mass spectrometry such as by 2D-LC/MS, for example as described herein) of the polypeptide may be shown to be between about 1.2-fold and 1.8-fold, for example at least about 1.2-, 1.3-, 1.4-, or 1.5-fold greater than the equivalent factor in a known non-potent (i.e., non-protective) reference vaccine, for example at least about 1.25-, 1.26-, 1.27-, 1.28, 1.29-, 1.30-, 1.35-, 1.30-, 1.41-, 1.42-, 1.43-, 1.44-, 1.45-, 1.46-, 1.47-, 1.48- or about 1.49-fold greater.

According to a second aspect of the invention, there is provided a Lipl32 polypeptide having the amino acid sequence SEQ ID NO:13, or an antigenic variant or portion thereof. A third aspect of the invention provides a nucleic acid encoding the polypeptide according to the second aspect of the invention, for example a nucleic acid having nucleotide sequence SEQ ID NO:45, or a complement thereof. Alternative functional variant nucleic acid sequences, related to SEQ ID NO:45 by degeneracy of the genetic code and still encoding SEQ ID NO:13, will be readily derivable by the skilled person. The polypeptide and/or nucleic acid may be isolated.

According to a fourth aspect of the invention, there is provided a vaccine composition comprising a Lipl32 epitope polypeptide having up to 250 amino acids and comprising a polypeptide according to the second aspect of the invention. The Lipl32 epitope polypeptide may, for example, have up to about 225, 200, 175, 150, 125, 100, 90, 80, 70, 60, 50, 40, 30 or up to about 20 amino acids. A protective concentration is a concentration correlated with vaccine compositions which are known to be protective, for example, giving a NSAF which is between 1.2- to 1.5-fold greater (e.g., about 1.27- or 1.49-fold greater) than in a known non-potent vaccine, as outlined above. A full-length Lipl32 epitope polypeptide, for example SEQ ID NO:1 from Leptospira serovar Canicola, may be excluded. The vaccine may be a subunit vaccine. The vaccine composition may comprise a polypeptide having amino acid sequence SEQ ID NO:13, or may comprise a polypeptide which is an antigenic variant or portion of SEQ ID NO:13. When the vaccine composition comprises SEQ ID NO:13 the concentration is at least 0.25 fmol SEQ ID NO:13 polypeptide per μg of vaccine protein, for example at least 0.5 or 1.0 or 1.2 fmol SEQ ID NO:13 polypeptide per μg of vaccine protein. The vaccine composition may comprise a nucleic acid according to the third aspect of the invention. The vaccine may be confirmed as a protective vaccine by a method according to the first aspect of the invention. The vaccine composition may be formulated in a pharmaceutically acceptable excipient or diluent and may further comprise a carrier and/or an adjuvant and/or a biologic response modifier.

The vaccine composition may be for use in a method of protecting an animal from infection by a bacterium of genus Leptospira. In a related aspect, there is provided a method of protecting an animal from infection by a bacterium of genus Leptospira comprising administering to the animal an effective amount of a vaccine composition according to the second aspect of the invention. The animal may be a mammal, such as a cow, dog, horse, sheep, pig, rodent or human being. The bacterium may be L. interrogans, L. kirschneri, L. noguchii, L. alexanderi, L. weilii, L. genomospecies 1, L. borgpetersenii, L. santarosai, L. kmetyi, L. canicola or L. icterohaemorragiae. The vaccine composition may be protective against infection against any one or more of these species.

According to a fifth aspect of the invention, there is provided a method of identifying an immunogenic element in a vaccine composition comprising submitting the composition to a two-dimensional liquid chromatography mass spectrometry (2D LC/MS) process.

The term “immunogenic element” indicates a polypeptide, protein or other compound which initiates an immune response in an animal or a cell exposed to the vaccine composition. Such an immune response may be assessed, for example, by detection of increased antibody expression and/or by a cell mediated immunity (CMI) assay.

The method may comprise the steps of
a) passing the vaccine composition through a strong cation exchange (SCX) column (for fractionation and simplification of proteins/peptides) and recovering elute from the column;
b) passing the elute from step (a) through an analytical reverse phase column and recovering elute from the column;
c) passing the elute from step (b) into a mass spectrometer and recording the output.

The method may further comprise an initial step of exposing the vaccine to a proteolytic enzyme to yield peptides for separation and sequencing, prior to passing the composition through the SCX column. Alternatively or additionally, the method may further comprise comparing the mass spectrum output from (c) to mass spectra information from a library of polypeptides and identifying a polypeptide in the library having a corresponding mass spectrum. Alternatively or additionally, the method may also further comprise a subsequent step of quantitation of target protein based on either peptides or intact protein using calibration curves prepared from synthetic peptides or proteins.

In a further alternative or additional embodiment, the method may further comprise subsequently obtaining a sample of the identified polypeptide and testing it for immunogenic properties. For example, once a polypeptide has been identified in the library it can readily be prepared by recombinant or other means by the skilled person, without use of inventive skill. The polypeptide can then be used, directly or as part of a vaccine composition, in an animal model to test for immunogenic properties, for example in a humoral cell response assay or a cell-mediated immunity assay, to determine whether the polypeptide induces an immune response. Methods such as those set out in Marbehant in European Pharmacopeia Forum. 1999 p. 11-16 can also be utilised to determine whether a polypeptide can be used to provide a protective vaccine composition. In the case where the polypeptide is identified as present and/or present at a certain concentration in more than one vaccine composition known to be protective, but not in vaccine compositions known to be non-protective, the polypeptide may be identified as a protective element in the composition, i.e., an element which produces an immune response in an animal such that the animal is protected from infection. Therefore, the method may be used to identify proteins or polypeptides as potential candidates for use as vaccines.

The present invention encompasses the polypeptides SEQ ID NOs:13-44 and functional variants thereof. In particular, it encompasses functional (or “antigenic”) variants of SEQ ID NO:13 and methods utilising these variant polypeptides. As used herein, a “variant” means a polypeptide in which the amino acid sequence differs from the base sequence from which it is derived in that one or more amino acids within the sequence are deleted or substituted with other amino acids. The variant is a functional variant, in that the functional characteristics of the polypeptide from which the variant is derived are maintained. For example, a similar immune response is elicited by exposure of an animal, or a sample from an animal, to the variant polypeptide, compared to the response elicited by exposure to the non-variant peptide. For example, an antibody which is known to be capable of binding to SEQ ID NO:13 will also be capable of binding to the variant polypeptide. In particular, any amino acid substitutions, additions or deletions must not alter or significantly alter the tertiary structure of one or more epitopes contained within the polypeptide from which the variant is derived, so that binding can still occur to an antibody known to be capable of binding to SEQ ID NO:13. The skilled person is readily able to determine appropriate functional variants and to determine the tertiary structure of an epitope and any alterations thereof, without the application of inventive skill.

Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

| Class | Amino acid examples |
|---|---|
| Nonpolar: | Ala, Val, Leu, Ile, Pro, Met, Phe, Trp |
| Uncharged polar: | Gly, Ser, Thr, Cys, Tyr, Asn, Gln |
| Acidic: | Asp, Glu |
| Basic: | Lys, Arg, His |

As is well known to those skilled in the art, altering the primary structure of a polypeptide by a conservative substitution may not significantly alter the activity of that polypeptide because the side-chain of the amino acid which is inserted into the sequence may be able to form similar bonds and contacts as the side chain of the amino acid which has been substituted out. This is so even when the substitution is in a region which is critical in determining the peptide's conformation.

As mentioned above, non-conservative substitutions are possible provided that these do not disrupt the tertiary structure of an epitope within the polypeptide, for example, which do not interrupt the immunogenicity (for example, the antigenicity) of the peptide.

Broadly speaking, fewer non-conservative substitutions will be possible without altering the biological activity of the polypeptide. Suitably, variants may be at least about 65% identical, about 70% identical, for example at least about 75% identical, such as at least about 80%, 85%, 90%, 91%, 92%, 93%, 94%, 95%, 96%, 97%, 98% or about 99% identical to the non-variant base sequence.

Sequence identity between amino acid sequences can be determined by comparing an alignment of the sequences. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties. The percentage sequence identity may be determined using BLAST sequence alignment software, publicly available via http://blast.ncbi.nlm.nih.gov/Blast.cgi (accessed on 13 Sep. 2010 and 13 Sep. 2011), using default parameter settings. Comparison should be determined for the full length sequence of the polypeptide, to avoid high sequence identity over a short fragment of the polypeptide.

A functional fragment of the polypeptide is a fragment wherein the functional characteristics of the polypeptide from which the fragment is derived are maintained, as described above. A functional fragment of SEQ ID NO:1 may comprise at least, for example, 8, 9, 10, 11, 12, 13, 14, 15, 16, 17, 18, 19 or 20 contiguous amino acids from within the sequence SEQ ID NO:l. For example, a functional fragment may comprise SEQ ID NO:13.

Using the standard genetic code, nucleic acids encoding the polypeptides may readily be conceived and manufactured by the skilled person. The nucleic acid may be DNA or RNA and, where it is a DNA molecule, it may for example comprise a cDNA or genomic DNA.

The invention encompasses nucleic acids (e.g., SEQ ID NO:45) encoding the polypeptide of the invention and variants thereof. The term “variant” or “functional variant” in relation to a nucleic acid sequences means any substitution of, variation of, modification of, replacement of deletion of, or addition of one or more nucleic acid(s) from or to a polynucleotide sequence providing the resultant polypeptide sequence encoded by the polynucleotide exhibits at least the same properties as the polypeptide encoded by the basic sequence. The term therefore includes allelic variants and also includes a polynucleotide (such as a complementary polynucleotide) which substantially hybridises to the polynucleotide sequence of the present invention. Such hybridisation may occur at or between low and high stringency conditions. In general terms, low stringency conditions can be defined a hybridisation in which the washing step takes place in a 0.330-0.825 M NaCl buffer solution at a temperature of about 40-48° C. below the calculated or actual melting temperature (Tₘ) of the probe sequence (for example, about ambient laboratory temperature to about 55° C.), while high stringency conditions involve a wash in a 0.0165-0.0330 M NaCl buffer solution at a temperature of about 5-10° C. below the calculated or actual Tₘ of the probe (for example, about 65° C.). The buffer solution may, for example, be SSC buffer (0.15M NaCl and 0.015M tri-sodium citrate), with the low stringency wash taking place in 3×SSC buffer and the high stringency wash taking place in 0.1×SSC buffer. Steps involved in hybridisation of nucleic acid sequences have been described for example in Sambrook et al. (1989; Molecular Cloning, Cold Spring Harbor Laboratory Press, Cold Spring Harbor).

Variant nucleic acids of the invention may be codon-optimised for expression in a particular host cell.

Polypeptides and nucleic acids of the invention may be prepared synthetically using conventional synthesisers. Alternatively, they may be produced using recombinant DNA technology and may be incorporated into suitable expression vector, which is then used to transform a suitable host cell, such as a prokaryotic cell such as E. coli. The transformed host cells are cultured and the polypeptide isolated therefrom. Vectors, cells and methods of this type form further aspects of the present invention.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

### BRIEF DESCRIPTION OF FIGURES

Embodiments of the invention will now be described, by way of example only, with reference to the following FIGS. 1-4 in which:

FIG. 1 shows strong cation exchange chromatograms of vaccines A-E recorded at 280 nm;

FIG. 2 shows vaccines A-E (lanes 2-6) run on a NU-PAGE gel stained with Coomassie Blue to detect protein (2A) and silver to detect LPS (2B), with lanes 1 and 9 containing a 3.5 kDa protein ladder and lanes 7-8 containing 500 ng and 10 μg E. coli LPS, respectively, as positive controls; FIGS. 2C and 2D show duplicate gels to 2A and 2B, respectively;

FIG. 3 shows the concentration of protein and LPS in Vaccines A-E (as sold) as determined by the Bradford and LAL assays, respectively. Two replicates of each vaccine were analysed for each assay; mean and standard deviation of the mean are shown; and

FIG. 4 shows concentrations of N and C terminus region tryptic peptides of Lipl32 in passed (n=2) and failed (n=2) vaccines (**=p≦0.01); three replicates of each vaccine were analysed.

### EXAMPLES

Methods

Culture of Leptospira interrogans serovar Canicola

Starter cultures were prepared by inoculation of 20 ml EMJH (Becton Dickinson, USA) media with 1 ml Leptospira interrogans serovar Canicola (strain Hond Utrecht IV) and incubated for 7 days at 30° C. with orbital agitation at 50 rpm. Larger working cultures (n=3) for proteome extraction were initiated by inoculation of 400 ml EMJH media with 10 ml of starter culture and incubated at 30° C. (50 rpm). Bacteria were harvested during the logarithmic growth phase (˜5*10⁸ cells/ml) by cooling the cultures on ice for 30 minutes and collection of cells by centrifugation at 4000×g for 20 minutes at 4° C. Growth was assessed by dark field microscopy using a Thoma counting chamber (0.1 mm depth, 1/400 m²). The bacterial cells were washed by suspension in 100 ml chilled phosphate buffered saline (PBS; 200 mM, pH 7.2) and pelleted (4000×g; 20 min, 4° C.) for subsequent protein extraction.

Extraction of L. Canicola proteins

Bacterial cell pellets were suspended in PBS (10 ml) containing PMSF (100 μM) and lysed by 6 second pulses of probe sonication (amplitude 60) using a Vibra-Cell ultrasonic processor (Sonics and Materials, USA) for 3 minutes on ice. Cell debris was removed by centrifugation at 300×g and the supernatant retained. A low speed cytosolic extract was produced from the supernatant by centrifugation at 32000×g for 30 minutes. The pellet was retained and the supernatant (cytosol extract) was then desalted by dilution with ammonium bicarbonate (2.5 mM; pH 8.0), concentrated to 0.5 ml by centrifugation in 5 kDa molecular weight cut off concentrators (Sartorius Stedim, France) and stored at −20° C.

The retained pellet was then washed by suspension in chilled phosphate buffered saline (PBS; 200 mM, pH 7.2) and collected by centrifugation (32000×g). The washed pellet was re-suspended in 3 ml lysis buffer (Urea 5 M, Thiourea 2 M, DTT 100 mM, CHAPS 2%, 3-(Decyldimethylammonio)propanesulfonate inner salt 2%, Pharmalytes 3-10 0.5%, Tris Base 0.48%) and centrifuged at 32000×g for 30 minutes. The insoluble protein fraction was precipitated in a 4-fold excess of ice cold acetone and incubated at −20° C. for 48 hours prior to centrifugation (3000×g for 30 minutes). The resulting pellet (insoluble fraction) was desalted and concentrated as before using ammonium bicarbonate and Vivaspin centrifugal filters respectively. Estimation of protein concentration for both fractions was then determined using the Bradford method (Sigma-Aldrich, UK), with bovine serum albumin as the calibration standard (0.05-1.0 mg/ml).

Vaccine Selection and Preparation

Bivalent vaccines, giving protection against L. canicola and L. icterohaemorragiae, from five different manufacturers were selected for analysis. Vaccine C is advertised to be of subunit manufacture comprising outer membrane proteins and vaccines A, B, D and E are derived from inactivated bacteria.

The vaccines were concentrated using 5 kDa molecular weight cut off concentrators, washed once with 2.5 mM Ammonium Bicarbonate and then concentrated again to a final volume of 0.5 ml. Estimation of protein in the vaccines was determined using the Bradford method with bovine serum albumin as the standard. Estimation of LPS in the vaccines was determined using an Endochrome K kit (Charles River, UK) which is based on the Limulus Amebocyte Lysate (LAL) assay.

Gel Electrophoresis

The protein and LPS content of the vaccines was determined by SDS-PAGE was conducted on the X cell surelock system (Invitrogen) using a 4-12% gradient NuPAGE gel at 150 W. Gels contained a 3.5 kDa protein ladder, two concentrations of LPS (Sigma) and aliquots (36 μl) of untreated vaccine (n=4); vaccine C was loaded at a lower volume (10 μl) due to its high protein content, larger volumes resulted in overloading of the gel.

LPS was visualised in the gel using a silver staining method [13, 30]; protein was visualised in an identical second gel using EZ run Coomassie stain (Fisher). In gel trypsin digestion was carried out according to the method described by Weeks [26]; mass analysis was performed as described previously on an Agilent 6520 Q-TOF using an acetonitrile gradient of 0% to 81% [v/v] over 21 minutes.

Strong Cation Exchange (SCX) Chromatography

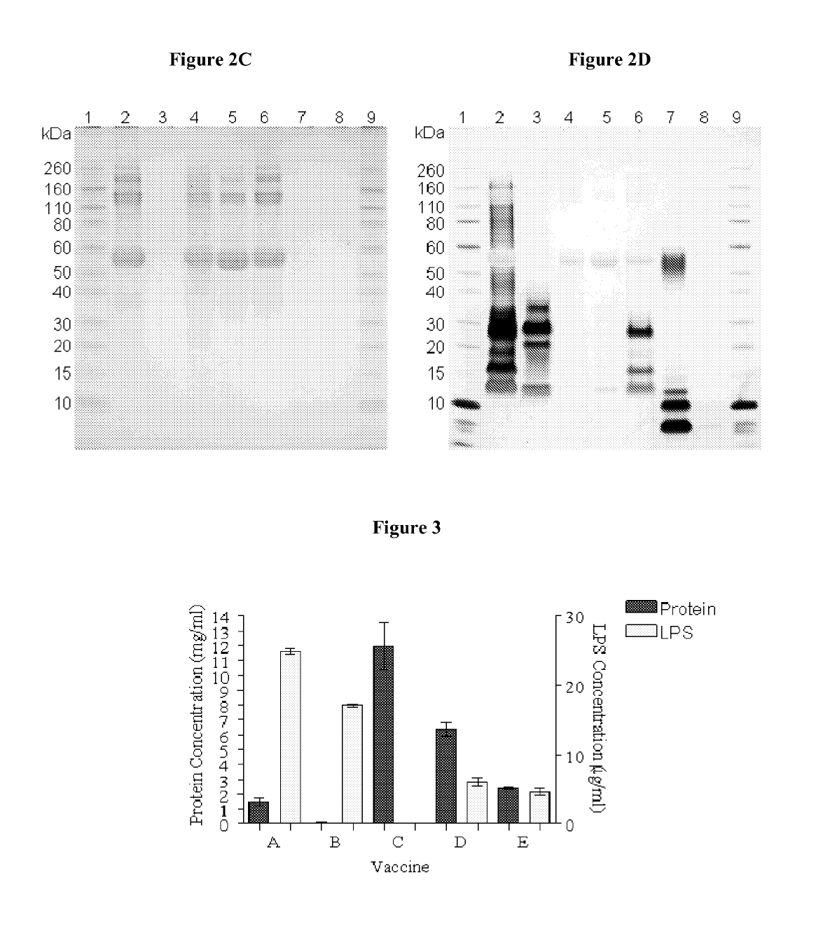

Three aliquots of each bacterial fraction and vaccine, normalised to 100 μg, were heat denatured at 95° C. for 5 minutes and then digested overnight with 2 μg sequencing grade trypsin (Promega). Digestion was terminated by the addition of 1 μl of 25.2M formic acid (Fluka). Due to the low concentration of protein found in vaccine B (FIG. 3 and Table 2) only 6 μg was used in each replicate.

Tryptic digests were centrifuged (5000×g for 1 minute) to remove cellular debris and the supernatant sampled to 250 μl HPLC vial inserts. Tryptic peptides (50 μl of digest) were chromatographed using a Hewlett-Packard 1100 system on a Biobasic SCX HPLC (2.1×100 mm) column (Thermo Scientific). Analytes were eluted at a flow rate of 0.25 ml/min with mobile phases comprising 75:25 2.5 mM ammonium acetate:acetonitrile pH 4.5 (A) and 75:25 250 mM ammonium acetate: acetonitrile pH 4.5 (B) and a binary gradient (t=0 min, A 100%; t=5 min, A 100%; t=18 min, 65% A; t=20 min, B 100%; t=22 min, A 100%; t=32 min, A 100%). The optical density of HPLC effluent was recorded at 280 nm and 15 fractions of 1 ml duration were collected between 8 and 23 min. The SCX fractions were taken to dryness at 60° C. using a centrifugal concentrator (Eppendorf 5301).

Reverse Phase Chromatography and Mass Analysis

Dried SCX fractions were dissolved in 0.1% v/v formic acid (20 μl) and analyzed on an Agilent 6520 Q-TOF mass spectrometer (Agilent Technologies) with an HPLC chip cube source. The chip consisted of a 40-nl enrichment column (Zorbax 300SB-C18 5 μm) and a 75 μm×150 mm separation column (Zorbax 300SB-C18 5 μm) driven by the Agilent Technologies 1200 series nano/capillary liquid chromatography system. Both systems were controlled by Masshunter Workstation Data Acquisition for Q-TOF (Version B.02.00, Patches 1,2; Agilent Technologies). Peptides were resuspended in 0.1% TFA with the chip switched to enrichment and using the capillary pump. After the sample volume (1 μl) passed through the enrichment column once, the chip was then switched to separation and peptides were eluted from the enrichment column and run through the separation column during a 47 minute gradient (4.5% to 72% [v/v] acetonitrile) directly into the mass spectrometer. The mass spectrometer was run in positive ion mode, with MS scans run over a range of m/z 100 to 3000 and at one spectra per second. Precursor ions were selected for auto MS/MS at an absolute threshold of 2000 and a relative threshold of 0.01, with a maximum of 5 precursors per cycle, active exclusion being set at 1 spectra and released after 3 minutes. Precursor charge-state selection and preference were set to 2+ and then 3+ and precursors were selected by abundance.

Bioinformatics

Spectrum Mill (Agilent, UK) and OMSSA (NCBI, USA) were used to extract MS/MS data from Masshunter acquisition files and proteins were subsequently identified by comparison of tryptic peptide product ion mass spectra against a protein database. Search parameters included selection of trypsin with up to two missed cleavage sites and a variable modification for oxidation of methionine residues. Identified proteins were exported as tab separated files for bioinformatics analysis. A protein database was used to determine optimal identification, these included the NCBI non-redundant (nr) database (ftp://ftp.ncbi.nlm.nih.gov/blast/db/FASTA accessed on 12 Jun. 2010 and 13 Sep. 2011), the uniprot sprot fasta database (ftp://ftp.uniprot.org/pub/databases/uniprot/knowledgebase accessed on 12 Jun. 2010 and 13 Sep. 2011) and a custom made database derived from chromosomes I and II of L. copenhageni (ftp://ftp.ncbi.nlm.nih.gov/genomes/Bacteria/Leptospira_{—}interrogans_serovar_{—}Copenhageni accessed on 12 Jun. 2010 or ftp://ftp.ncbi.nlm.nih.gov/genomes/Bacteria/Leptospira_{—}interrogans_serovar_{—}Copenhageni_Fiocruz_L1_{—}130_uid58065/ accessed on 13 Sep. 2011). The Uniprot database identified 59% less proteins than the custom database and was therefore disregarded for further use. The non-redundant NCBI database recognized 29% more proteins; however, the overall number of Leptospira identifications was less and certain proteins, identified in the custom database, were noticeably absent, suggesting that the non-redundant database did not contain the entire L. copenhageni proteome. The custom database therefore was utilised for subsequent mass spectra searches (unless specified otherwise).

The false discovery rate (FDR) [11] was calculated by searching the three replicates of vaccine A against reverse and forward-reverse decoy databases, created from the L. copenhageni proteome using the Perl script decoy.pl available from (http://www.matrixscience.com/help/decoy_help.html accessed on 5 Aug. 2010 and 13 Sep. 2011). The FDR for OMSSA was calculated as 6.1±3% (mean±1 SD) whereas the FDR for Spectrum Mill was 2.0±1%.

The relative abundance of proteins present in all three technical repeats was determined through spectral counting [25]. With the normalised spectrum abundance factor (NSAF) used to account for differences in peptide length, allowing for accurate comparison of coverage between individual vaccines. The normalised spectrum abundance factor (NSAF), calculated as described by Zybailov et al. [12], was used to account for differences in peptide length, allowing for comparison of coverage between individual vaccines, using a novel program developed at the Animal Health and Veterinary Laboratories Agency.

Assessment of Performance

To ensure accuracy and reproducibility of chromatographic separations, performance was monitored by the analysis of specific standards prior to each run. SCX was monitored by using tryptically digested BSA (25 μg/ml) and two blanks to control for sample carryover. RP-HPLC and subsequent mass spectrometry was assessed by monitoring the retention time and intensity of ions derived from a synthetic peptide mix (10 ng/ml) containing the polypeptides:

(SEQ ID NO: 2)
MRFA
(SEQ ID NO: 3)
AVDQLNEQSSEPNIYR
(SEQ ID NO: 4)
ARPQELPFLASIQNQGR
(SEQ ID NO: 5)
NYINQYSEVAIQMVMHMQPK
(SEQ ID NO: 6)
ISVNNVLPVDFNLMQQK
(SEQ ID NO: 7)
VTALYEGFTVQNEANK

Lipl32 Quantitation

Synthetic peptide analogues, corresponding to N and C terminal region tryptic peptides found in Lipl32 (see below and Table 5), were obtained (Peptides Synthetics, UK) for detection and quantification using multiple reaction monitoring (Table 1). Optimal transitions and conditions for the peptides of interest (Table 1) were obtained using the MS and MS/MS data from previous Q-Tof analysis of vaccine C. The acquired data was quantified using the Agilent Masshunter Quantitative Analysis software (Version B.03.01; Agilent Technologies).

TABLE 1
Tryptic peptides and product ions selected for detection and
quantification by MS using multiple selection reaction monitoring.

| | | | Precursor | Product | Retention |
|---|---|---|---|---|---|
| | | Molecular | ion for MRM | ion for MRM | Time |
| Peptide Sequence | Terminus | Mass (Da) | (m/z) | (m/z) | (min) |
| SSFVLSEDTIPGTNETVK | N | 1924.07 | 962.50 | 845.40 | 13.16 |
| (SEQ ID NO: 13) | | | | | |
| ISFTTYKPGEVK | C | 1369.56 | 457.30 | 628.80 | 10.80 |
| (SEQ ID NO: 14) | | | | | |

Passed (n=2) and failed (n=2) batches of vaccine C were prepared, quantitated and digested as described above. SCX separation was performed generally as described above, with some changes. The 15 fractions of each replicate were recombined prior to being taken to dryness, redissolved in 0.1% v/v formic acid (50 μl) and analyzed on an Agilent 6410 Triple Quadrupole mass spectrometer (Agilent Technologies) with an HPLC chip cube source. The chromatography chip consisted of a 160-nl enrichment column (Zorbax 300 SB-C18 5 μm) and a 75 μm×150 mm analytical column (Zorbax 300 SB-C18 5 μm) driven by the Agilent Technologies 1200 series nano/capillary liquid chromatography system. Both systems were controlled by Masshunter Workstation Data Acquisition for Triple Quadrupole (Version B.02.01; Agilent Technologies). Tryptic peptides (1 μl) were loaded onto the enrichment column of the chip and washed eight times with 0.1% TFA. Peptides were then separated on the analytical column using an acetonitrile gradient (4.5% to 90% [v/v]) over 25 minutes and eluted directly into the mass spectrometer. The mass spectrometer was run in positive ion mode, with the electrospray voltage set to 1900V and gas temperature and pressure set at 300° C. and 4 l/min respectively.

Results

Protein Identification

Initial analysis of vaccines using 2D-LC/MS revealed a high level of albumin, when searched against the NCBI non-redundant database, which could mask the presence of low abundant proteins. A number of different approaches were attempted to minimise albumin identification including filter aided washing during sample preparation and the use of an albumin exclusion list. It was found that washing the sample once improved identification of lower abundant proteins dramatically, additional washes were attempted but these led to a reduction in overall proteins identified; the use of an albumin exclusion list had no effect, most likely due to factors such as the length of the exclusion list and the speed of the instrument.

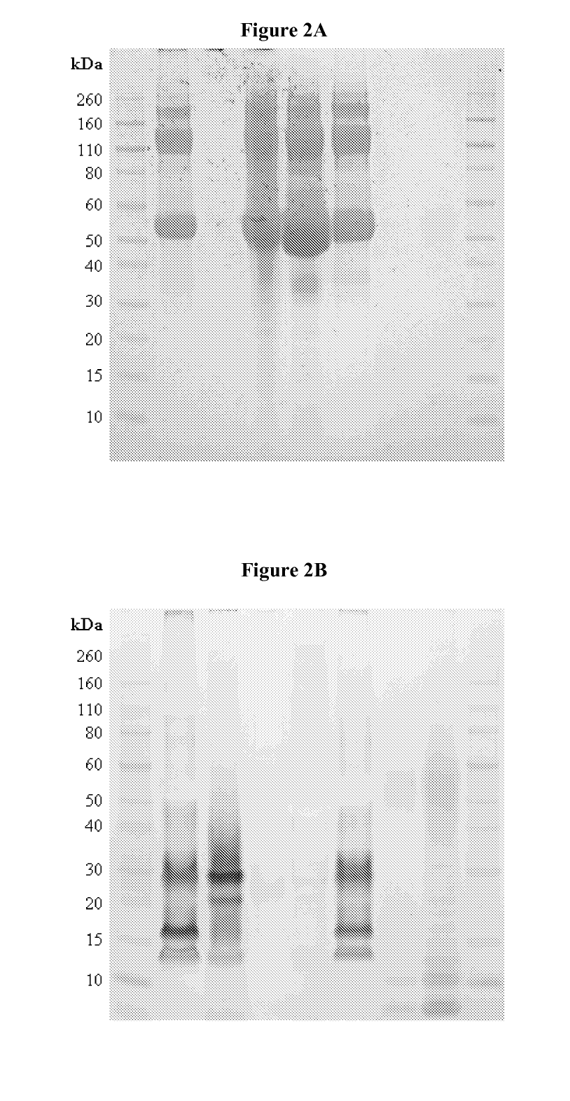

The amount of protein present in 1 ml single dose aliquots (as sold) of vaccine is shown in Table 2 and good correlation was observed with the SDS gels in FIG. 2.

TABLE 2
Concentration of protein in Vaccines A-E (as sold)
as determined by the Bradford assay

| | | Concentration |
|---|---|---|
| | | (mg; mean ± 1 SD) |
| Vaccine | N | Protein |
| A | 2 | 1.47 ± 0.27 |
| B | 2 | 0.07 ± 0.01 |
| C | 2 | 11.94 ± 1.57 |
| D | 2 | 6.38 ± 0.44 |
| E | 2 | 2.41 ± 0.06 |

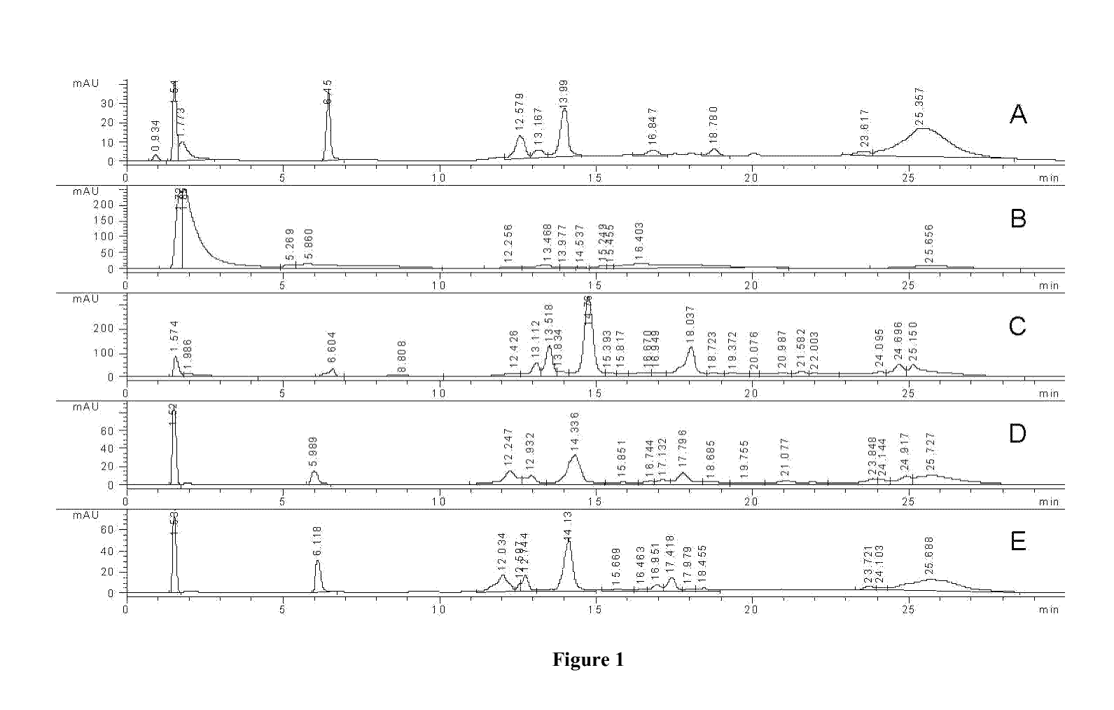

No protein was observed in vaccine B. However, when separated via strong cation exchange chromatography (FIG. 1), definite peaks were observed; subsequent mass spectrometry identified 8 proteins (see also Table 3A listing 6 of these protein genes; accession numbers are NCBI GI numbers), suggesting that the vaccine may be peptide based rather than protein.

The chromatograms for vaccines A and C-E show fairly similar profiles with an intense peak at ˜14 minutes appearing in each, this is most likely the active element(s) of the vaccines.

Further Characterisation Reveals some Vaccines to be Free from LPS

Gram negative bacteria such as Leptospira contain LPS within their cell wall, which can be highly immunogenic. As the majority of the vaccines analysed were derived from inactivated bacteria the level of LPS in each was quantified to help determine the extent of its involvement in the host.

Gel electrophoresis (FIG. 2) showed vaccines C and D to contain a complete absence of detectable LPS but reasonably high levels of protein suggesting that these vaccines rely on protein alone for their immunogenicity. An LPS-specific silver stain (FIGS. 2B and 2D), which works through interaction with the polysaccharide part of the molecule [27], showed that vaccine C did not contain any detectable LPS. For confirmation the LAL assay (FIG. 3), which functions by interaction with the lipid A [28] and core region [29] of LPS, was also performed on vaccines A-E. LPS was not detectable in vaccine C using the LAL assay either, suggesting that it is absent in its entirety from the vaccine.

Analysis of Vaccine Proteomes allows for Identification of Conserved Proteins

Identification of vaccine proteins required that mass data be interrogated against a database using a suitable search program. For the purposes of this study Spectrum Mill and OMSSA were chosen, mass spectra was analysed using equivalent conditions. Proteins which were common to each vaccine, across all three technical repeats, were then determined using Microsoft Access and their respective abundances calculated using the normalised spectrum abundance factor (Table 3A). This showed that Lipl32 was present in all vaccines. Table 3B shows an optimised analysis of the results, which also showed that Flagellin protein was present in all vaccines.

The number of proteins identified using OMMSA was substantially higher in vaccines C-E than the number identified using Spectrum Mill, which is consistent with the false discovery rates calculated for the two programs; it was therefore decided that the results gained through Spectrum Mill were more reliable and this was used in subsequent analyses.

TABLE 3A
Showing conserved proteins present in commercially available vaccines from five different
manufacturers (A-E). Higher ln(NSAF) values indicate greater abundance.

| Accession | | Mean ln(NSAF) | | | | | | | | | |
|---|---|---|---|---|---|---|---|---|---|---|---|
| Number | Gene Name | A* | A^{†} | B* | B^{†} | C* | C^{†} | D* | D^{†} | E* | E^{†} |
| 45658793 | LipL41 | −2.85 | −3.46 | ND | ND | −2.19 | −2.95 | −2.03 | −3.12 | −2.23 | −3.92 |
| | (SEQ ID NO: 8) | | | | | | | | | | |
| 45657753 | Flagellin Protein | −3.2 | −2.63 | ND | ND | −3 | −3.19 | −2.21 | −3.14 | −2.54 | −3.52 |
| | (SEQ ID NO: 9) | | | | | | | | | | |
| 45657230 | LipL32 | −3.45 | ND | −2.19 | ND | −2.37 | ND | −2.16 | ND | −2.77 | ND |
| | (SEQ ID NO: 1) | | | | | | | | | | |
| 45656175 | Cell Wall | −3.58 | ND | ND | ND | −2.29 | ND | −2.23 | ND | −2.89 | ND |
| | Hydrolase | | | | | | | | | | |
| | (SEQ ID NO: 10) | | | | | | | | | | |
| 45656311 | Hypothetical Protein | −4.15 | ND | ND | ND | −3.01 | ND | −2.81 | ND | −2.78 | ND |
| | LIC10411 | | | | | | | | | | |
| | (SEQ ID NO: 11) | | | | | | | | | | |
| 45656623 | Hypothetical Protein | ND | −3.03 | ND | ND | ND | −1.67 | ND | −0.89 | ND | −1.61 |
| | LIC10725 | | | | | | | | | | |
| | (SEQ ID NO: 12) | | | | | | | | | | |

| | | | | | | | | | | |
|---|---|---|---|---|---|---|---|---|---|---|
| Mean Total Proteins ± 1 SD | 86 ± 21 | 85 ± 20 | 8 ± 8 | 6 ± 8 | 31 ± 4 | 82 ± 14 | 19 ± 4 | 44 ± 14 | 30 ± 13 | 50 ± 7 |

*= Data obtained through searching spectra using Spectrum Mill
^{†}= Data obtained through searching spectra using OMSSA
ND = Protein not detected using this program

TABLE 3B
Conserved proteins present in commercially available vaccines
from five different manufacturers (A-E).
Higher ln(NSAF) values indicate greater abundance.

| Accession | | Mean ln(NSAF) | | | | |
|---|---|---|---|---|---|---|
| Number | Gene Name | A | B | C | D | E |
| 45657230 | LipL32 | −4.11 | −2.64 | −2.48 | −2.22 | −2.88 |
| 45657753 | Flagellin Protein | −3.41 | −2.44 | −3.01 | −2.35 | −2.59 |
| 45658793 | LipL41 | −3.35 | ND | −2.09 | −2.08 | −2.39 |
| 45656311 | Hypothetical | −2.45 | ND | −3.13 | −2.96 | −3.03 |
| | Protein LIC10411 | | | | | |

| | | | | | |
|---|---|---|---|---|---|
| Mean Proteins ± 1 SD | 221 ± 31 | 9 ± 8 | 34 ± 4 | 21 ± 5 | 34 ± 17 |

ND = Protein not detected using this program

Two batches of a vaccine, one which had passed the in vivo potency test (a) and another which had failed (β), were subsequently analysed using 2D-LC/MS (Table 4A; accession numbers are NCBI GI numbers). Lipl32, which had previously been identified as being present in vaccines A-E (Tables 2A & 2B), is shown to be present at a higher abundance (p<0.01) in the passed batch (a) compared to the failed batch (β), with an NSAF 1.49-fold higher.

TABLE 4A
Showing conserved proteins present in two different batches of the same vaccine, the first
(α) passed an in vivo potency test whereas the second (β) failed.

| | | Mean | | |
|---|---|---|---|---|
| Accession | | ln(NSAF) | | |
| Number | Gene Name | α | β | P value |
| 45657309 | Histidine Kinase Sensor Protein | −4.71 | −4.37 | 5.45E−3 |
| 45657230 | LipL32 | −2.78 | −3.18 | 8.13E−3 |
| 45657022 | Hypothetical Protein LIC11138 | −4.96 | −4.39 | 9.22E−2 |
| 45658246 | Isoleucyl-tRNA Synthetase | −4.52 | −3.81 | 1.15E−1 |
| 45658793 | LipL41 | −2.48 | −2.26 | 2.01E−1 |
| 45655648 | Acyl Carrier Protein | −2.59 | −2.25 | 2.26E−1 |
| 45656175 | Cell Wall Hydrolase | −1.85 | −1.69 | 3.14E−1 |
| 45658886 | LipL36 | −4.65 | −4.31 | 4.02E−1 |
| 45657930 | Cysteine Synthase | −4.11 | −4.37 | 4.14E−1 |
| 45657213 | Chaperonin GroEL | −3.18 | −3.29 | 4.50E−1 |
| 45657748 | Putative Lipoprotein | −3.80 | −4.06 | 4.93E−1 |
| 45657753 | Flagellin Protein | −3.65 | −3.91 | 5.10E−1 |
| 45656611 | Putative Lipoprotein | −3.72 | −3.59 | 5.69E−1 |
| 45658988 | Hypothetical Protein LIC13166 | −3.49 | −3.63 | 6.12E−1 |
| 45657078 | Putative Citrate Lyase | −4.01 | −3.97 | 9.12E−1 |

| | | |
|---|---|---|
| Mean Total Proteins ± 1 SD | 42 ± 2 | 34 ± 2 |

Table 4B shows the results after an optimised analysis, in which Lipl32 was again shown to be present at a higher abundance (p≦0.05) in the passed batch, with an NSAF 1.27-fold higher.

TABLE 4B
Showing conserved proteins present in two different batches of the same vaccine, the first
passed an in vivo potency test whereas the second failed.

| Accession | | Mean ln(NSAF) | | |
|---|---|---|---|---|
| Number | Gene Name | Passed | Failed | P Value |
| 45657309 | Histidine Kinase Sensor Protein | −4.92 | −4.57 | 9.23E−03 |
| 45657230 | LipL32 | −2.83 | −3.07 | 3.20E−02 |
| 45658246 | Isoleucyl-tRNA Synthetase | −4.46 | −3.89 | 8.58E−02 |
| 45658988 | Hypothetical Protein LIC13166 | −3.54 | −3.7 | 9.39E−02 |
| 45656611 | Putative Lipoprotein | −3.94 | −3.63 | 1.05E−01 |
| 45657748 | Putative Lipoprotein | −3.78 | −4.27 | 1.70E−01 |
| 45658793 | LipL41 | −2.65 | −2.44 | 1.90E−01 |
| 45656945 | Cell Division Protein | −5.68 | −5.24 | 2.04E−01 |
| 45657213 | Chaperonin GroEL | −3.22 | −3.35 | 3.63E−01 |
| 45655648 | Acyl Carrier Protein | −2.71 | −2.36 | 3.65E−01 |
| 45658886 | LipL36 | −4.86 | −4.51 | 3.76E−01 |
| 45657869 | ATP-dependent Protease | −4.58 | −4.73 | 3.83E−01 |
| 45657930 | Cysteine Synthase | −4.33 | −4.58 | 4.10E−01 |
| 45657753 | Flagellin Protein | −3.79 | −4.12 | 4.66E−01 |
| 45656175 | Cell Wall Hydrolase | −1.97 | −1.88 | 5.42E−01 |
| 45657078 | Putative Citrate Lyase | −3.95 | −3.91 | 8.55E−01 |

| | | |
|---|---|---|
| Mean Total Proteins ± 1 SD | 54 ± 8 | 42 ± 4 |

Spectrum Mill was subsequently used to identify which Lipl32 peptides following digestion with trypsin (i.e., tryptic peptides) were present in the two batches, allowing comparison with those found in L. Canicola, as shown in Table 5.

TABLE 5
Comparison of tryptic peptides present in an in silico
digest of Lipl32 against Lipl32 tryptic peptides identified in
L. Canicola and two batches of vaccine C.

| SEQ ID | | | Passed | Failed |
|---|---|---|---|---|
| NO. | in silico Digest | L. Canicola | Vaccine | Vaccine |
| 15 | AYYLYVWIPAVIAEMGVR | + | − | − |
| 16 | IPNPPK | + | − | − |
| 17 | LDDDDDGDDTYK | + | − | − |
| 18 | SMPHWFDTWIR | + | + | + |
| 13 | SSFVLSEDTIPGTNETVK | + | + | − |
| 19 | IKIPNPPK | + | − | − |
| 14 | ISFTTYKPGEVK | + | − | + |
| 20 | MISPTGEIGEPGDGDLVSDAFK | + | − | − |
| 21 | MSAIMPDQIAK | + | + | + |
| 22 | AAKAKPVQK | − | − | − |
| 23 | AATPEEKSMPHWFDTWIR | − | − | − |
| 24 | AKPVQKLDDDDDGDDTYK | − | − | − |
| 25 | EERHNK | − | − | − |
| 26 | GSFVASVGLLFPPGIPGVSPLIHSNPEELQK | − | − | − |
| 27 | IPNPPKSFDDLK | − | − | − |
| 28 | KAYYLYVWIPAVIAEMGVR | − | − | − |
| 29 | KLSILAISVALFASITACGAFGGLPSLK | − | − | − |
| 30 | LDDDDDGDDTYKEER | − | − | − |
| 31 | LLVRGLYR | − | − | − |
| 32 | LSILAISVALFASITACGAFGGLPSLK | − | − | − |
| 33 | QAIAAEESLK | − | − | − |
| 34 | QAIAAEESLKK | − | − | − |
| 35 | SFDDLKNIDTK | − | − | − |
| 36 | SMPHWFDTWIRVER | − | − | − |
| 37 | TLLPYGSVINYYGYVKPGQAPDGLVDGNK | − | − | − |
| 38 | TLLPYGSVINYYGYVKPGQAPDGLVDGNKK | − | − | − |
| 39 | YNSLTRIK | − | − | − |
| 40 | GLYRISFTTYKPGEVK | − | − | − |
| 41 | HNKYNSLTR | − | − | − |
| 42 | MISPTGEIGEPGDGDLVSDAFKAATPEEK | − | − | − |
| 43 | MSAIMPDQIAKAAK | − | − | − |
| 44 | VERMSAIMPDQIAK | − | − | − |

Quantitation of Lipl32 in Passed and Failed Batches of Vaccine

To enable accurate determination of the concentration of Lipl32 in the vaccines, two tryptic peptides from Table 5 (SEQ IS NOs:13 & 14), corresponding to regions in the N and C termini of the protein, were selected for quantitation using multiple reaction monitoring (MRM). The limit of detection (LoD) of the N and C termini region peptides was determined [30] as 0.025 and 0.018 fmol/μg respectively, using the empirical method:

LoD=LoB+1.645(SD_{low concentration sample})

where LoB=mean_{blank}+1.645(SD_{blank})

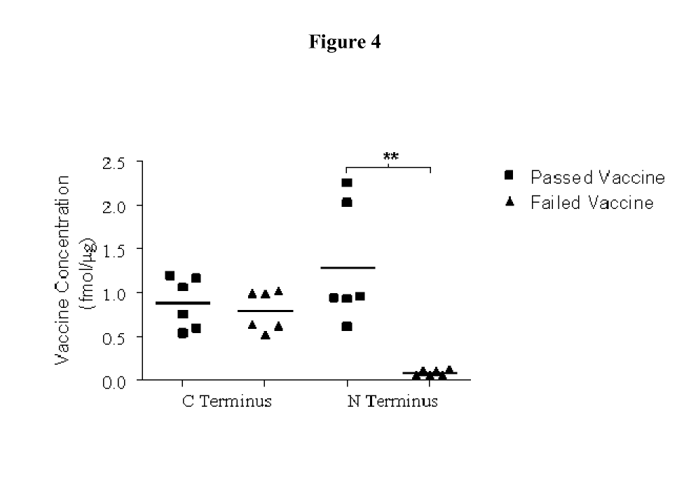

The limit of quantification (LOQ), defined as the lowest concentration of the standard with a coefficient of variation lower than 20% [30], was 0.02 and 0.05 fmol/μg for the N and C termini region peptides. Calibration curves for both peptides were constructed using Masshunter software (Agilent, UK) over the tested concentration range (0.01-100 fmol/μg) allowing determination of sample concentration (FIG. 4). The concentration of the N terminus region region (SEQ ID NO:13) was significantly higher (p≦0.01) in passed batches (n=2) of vaccine compared to failed (n=2); no statistical difference was observed in the concentration of the C terminus (SEQ ID NO:14) region.

Discussion

Given that vaccines A, B, D and E were derived from inactivated forms of the same bacteria, proteomic analysis reveals an unexpected heterogeneity of components. This, together with an absence of detectable LPS in vaccines C and D, suggests stark contrasts in the purification methods employed during manufacture. It seems improbable that the constituent responsible for protective immunity would be different in each vaccine and, since these vaccines are all known to be equally efficacious, the inventors concluded that they must share some common immunogenic element. The absence of detectable LPS in vaccine C rules it out as a potential target molecule for an in vitro vaccine potency test.

The proteomes of the five vaccines were, therefore, compared against one another. Comparison of the vaccines which showed a positive reaction to the protein assay (i.e. A and C-E) revealed five conserved proteins. Of these it was the flagellin and lipoproteins that were of interest, as both are known to be potential immunogens[14, 31]. When vaccine B was included in the comparison, the only proteins identified as being conserved were flagellin and Lipl32, which is noteworthy as they were also found in a high abundance in the bacteria itself.

Lipl32, also known as hemolysis associated protein-1 (Hap-1), is a major surface expressed outer membrane protein [16] found in pathogenic Leptospira species [17]. It is known to provide cross protection against Leptospira interrogans [18] and various different methods for presenting it as a vaccine have been trialled [19, 20]. It is interesting to note that it is present in batches of vaccine which do not elicit an immune reaction, albeit at a lower abundance, indicating that a certain profusion of the protein, when measured by 2D-LC/MS after tryptic enzyme treatment, is required for a vaccine to have the desired effect.

Initial analysis of passed and failed batches of vaccine using spectral counting (NSAF) estimated that Lipl32 was present at a statistically (p≦0.05) higher abundance in passed vaccines compared to failed indicating that it would make a good target molecule for an in vitro potency test. To corroborate this, multiple reaction monitoring (MRM) was used to accurately quantitate [32] the concentration of Lipl32 in passed (n=2) and failed (n=2) batches of vaccine C. MRM quantitates proteins based on specific peptide sequences; to ensure good coverage of Lipl32 two peptide sequences, corresponding to start (N terminus) and end (C terminus) regions of the protein, were selected for quantitation. The concentration of the C terminus peptide was approximately the same across both conditions (FIG. 4). However, the concentration of the N terminus peptide was substantially (p≦0.01) higher in passed batches. This suggests that Lipl32 is somehow truncated in failed batches of vaccine, which would result in fewer spectra being detected during Q-TOF analysis accounting for the lower abundance observed through spectral counting. The truncation of Lipl32 in failed batches indicates that this missing region of protein may play an important role in conveying protective immunity to the host.

In conclusion, the inventors have shown 2D-LC/MS to be an effective tool for vaccine analysis and has identified a target protein, Lipl32, upon which to base an in vitro potency test, in which the NSAF is significantly higher in potent vaccines than in non-potent vaccines. N terminal amino acid quantitation of Lipl32 has been shown to be a highly quantitative, novel assay for the determination of potency in vitro.

### REFERENCES

1. Adler, B. and A. de la Pena Moctezuma, Leptospira and leptospirosis. Vet Microbiol, 2010. 140(3-4): p. 287-96.
2. Levett, P. N., Leptospirosis. Clin Microbiol Rev, 2001. 14(2): p. 296-326.
3. Defra, Zoonoses Report: United Kingdom. 2008.
4. Koizumi, N. and H. Watanabe, Leptospirosis vaccines: past, present, and future. J Postgrad Med, 2005. 51(3): p. 210-4.
5. Klaasen, H. L., et al., Duration of immunity in dogs vaccinated against leptospirosis with a bivalent inactivated vaccine. Vet Microbiol, 2003. 95(1-2): p. 121-32.
6. Marbehant, P., Alternatives to animal challenge tests in the batch control of Leptospira vaccines for Veterinary Use, in European Pharmacopeia Forum. 1999. p. 11-16.
7. Woodward, M. J., Leptospira, in Molecular Medical Microbiology, M. Sussman, Editor. 2001, Academic Press. p. 2137-2158
8. Silva, E. F., et al., Characterization of virulence of Leptospira isolates in a hamster model. Vaccine, 2008. 26(31): p. 3892-6.
9. Ebert, E., Alternatives to animal challenge tests in the batch control of Leptospira vaccines for Veterinary Use, in European Pharmacopeia Forum. 1999. p. 102-109.
10. Ruby, K. W., Alternatives to animal challenge tests in the batch control of Leptospira vaccines for Veterinary Use, in European Pharmacopeia Forum. 1999. p. 35-45.
11. Elias, J. E., et al., Comparative evaluation of mass spectrometry platforms used in large-scale proteomics investigations. Nat Meth, 2005. 2(9): p. 667-675.
12. Zybailov, B., et al., Statistical analysis of membrane proteome expression changes in Saccharomyces cerevisiae. J Proteome Res, 2006. 5(9): p. 2339-47.
13. Tsai, C. M. and C. E. Frasch, A sensitive silver stain for detecting lipopolysaccharides in polyacrylamide gels. Anal Biochem, 1982. 119(1): p. 115-9.
14. Haake, D. A., et al., Leptospiral outer membrane proteins OmpL1 and LipL41 exhibit synergistic immunoprotection. Infect Immun, 1999. 67(12): p. 6572-82.
15. Hayashi, F., et al., The innate immune response to bacterial flagellin is mediated by Toll-like receptor 5. Nature, 2001. 410(6832): p. 1099-1103.
16. Cullen, P. A., et al., Surfaceome of Leptospira spp. Infect Immun, 2005. 73(8): p. 4853-63.
17. Haake, D. A., et al., The leptospiral major outer membrane protein LipL32 is a lipoprotein expressed during mammalian infection. Infect Immun, 2000. 68(4): p. 2276-85.
18. Branger, C., et al., Identification of the hemolysis-associated protein 1 as a cross-protective immunogen of Leptospira interrogans by adenovirus-mediated vaccination. Infect Immun, 2001. 69(11): p. 6831-8.
19. Branger, C., et al., Protection against Leptospira interrogans sensu lato challenge by DNA immunization with the gene encoding hemolysin-associated protein 1. Infect Immun, 2005. 73(7): p. 4062-9.
20. Seixas, F. K., et al., Evaluation of different ways of presenting LipL32 to the immune system with the aim of developing a recombinant vaccine against leptospirosis. Can J Microbiol, 2007. 53(4): p. 472-9.
21. Sakolvaree, Y., et al., Proteome and immunome of pathogenic Leptospira spp. revealed by 2DE and 2DE-immunoblotting with immune serum. Asian Pac J Allergy Immunol, 2007. 25(1): p. 53-73.
22. Malmstrom, J., et al., Proteome-wide cellular protein concentrations of the human pathogen Leptospira interrogans. Nature, 2009. 460(7256): p. 762-5.
23. Dong, H., et al., Characterization of the ompL1 gene of pathogenic Leptospira species in China and cross-immunogenicity of the OmpL1 protein. BMC Microbiol, 2008. 8: p. 223.
24. Wang, Z., L. Jin, and A. Wegrzyn, Leptospirosis vaccines. Microb Cell Fact, 2007. 6: p. 39.
25. Zybailov, B., et al., Correlation of relative abundance ratios derived from peptide ion chromatograms and spectrum counting for quantitative proteomic analysis using stable isotope labeling. Anal Chem, 2005. 77(19): p. 6218-24.
26. Weeks, M. E., Urinary proteome profiling using 2D-DIGE and LC-MS/MS. Methods Mol Biol, 2010. 658: p. 293-309.
27. Fomsgaard, A., M. A. Freudenberg, and C. Galanos, Modification of the silver staining technique to detect lipopolysaccharide in polyacrylamide gels. J Clin Microbiol, 1990. 28(12): p. 2627-31.
28. Takayama, K., et al., Influence of fine structure of lipid A on Limulus amebocyte lysate clotting and toxic activities. Infect Immun, 1984. 45(2): p. 350-5.
29. Conrad, M. L., R. L. Pardy, and P. B. Armstrong, Response of the blood cell of the American horseshoe crab, Limulus polyphemus, to a lipopolysaccharide-like molecule from the green alga Chlorella. Biol Bull, 2001. 201(2): p. 246-7.
30. Armbruster, D. A. and T. Pry, Limit of blank, limit of detection and limit of quantitation. Clin Biochem Rev, 2008. 29 Suppl 1: p. S49-52.
31. Hauk, P., et al., In LipL32, the major leptospiral lipoprotein, the C terminus is the primary immunogenic domain and mediates interaction with collagen IV and plasma fibronectin. Infect Immun, 2008. 76(6): p. 2642-50.
32. Ong, S. E. and M. Mann, Mass spectrometry-based proteomics turns quantitative. Nat Chem Biol, 2005. 1(5): p. 252-62.

1. A method of identifying a protective Leptospira vaccine composition, comprising determining that the composition contains a protective concentration of a Lipl32 epitope polypeptide.

2. The method of claim 1 in which a Normalised Spectrum Abundance Factor is measured for the Lipl32 epitope polypeptide and found to be higher in the protective vaccine composition than in a known non-protective vaccine composition.

3. The method of claim 1 comprising determining that the composition contains a protective concentration of a polypeptide comprising SEQ ID NO:13 or an antigenic variant or portion thereof.

4. The method of claim 1 wherein the Leptospira vaccine composition comprises an attenuated Leptospira species.

5. The method of claim 4 wherein the attenuated Leptospira species is L. interrogans, L. kirschneri, L. noguchii, L. alexanderi, L. weilii, L. genomospecies 1, L. borgpetersenii, L. santarosai, L. kmetyi, L. canicola or L. icterohaemorragiae.

6. The method of claim 1 wherein the Lipl32 epitope polypeptide is SEQ ID NO:13.

7. The method of claim 6 wherein the protective concentration of Lipl32 epitope polypeptide is at least 0.25_fmol/μg vaccine protein.

8. The method of claim 7 wherein the protective concentration of Lipl32 epitope polypeptide is at least 0.5_fmol/μg vaccine protein.

9. The method of claim 1 wherein the Lipl32 epitope polypeptide is Lipl32 (SEQ ID NO:1).

10. The method of claim 1 comprising submitting the composition to a two-dimensional liquid chromatography mass spectrometry (2D LC/MS) process.

11. A Lipl32 epitope polypeptide having the sequence SEQ ID NO:13, or an antigenic variant or portion thereof.

12. A nucleic acid encoding the polypeptide of claim 11, or a complement or functional variant of such a nucleic acid.

13. A vaccine composition comprising a polypeptide having up to 250 amino acids and comprising a polypeptide according to claim 11.

14. A vaccine composition comprising a nucleic acid of claim 12.

15. The vaccine composition of claim 13 for use in a method of protecting an animal from infection by a bacterium of genus Leptospira.

16. (canceled)

17. The vaccine composition of claim 15 wherein the animal is a mammal.

18. The vaccine composition of claim 17 wherein the mammal is a cow, dog, horse, sheep, pig, rodent or human being.

19. The vaccine composition of claim 15 wherein the bacterium is L. interrogans, L. kirschneri, L. noguchii, L. alexanderi, L. weilii, L. genomospecies 1, L. borgpetersenii, L. santarosai, L. kmetyi, L. canicola or L. icterohaemorragiae.

20. A method of identifying an immunogenic element in a vaccine composition comprising submitting the composition to a two-dimensional liquid chromatography mass spectrometry (2D LC/MS) process.

21. The method of claim 20 comprising the steps of
a. passing the vaccine composition through a strong cation exchange (SCX) column and recovering elute from the column;
b. passing the elute from step (a) through an analytical reverse phase column and recovering elute from the column; and
c. passing the elute from step (b) into a mass spectrometer and recording the output.

22. The method of claim 21 wherein the mass spectrum output from (c) is compared to mass spectra information from a library of proteins and identifying a protein in the library having a corresponding mass spectrum.

23. The method of claim 22 comprising subsequently obtaining a sample of the identified protein and testing it for immunogenic properties.

24. The method of claim 20 which is used to analyse vaccine compositions and the results used to identify proteins or polypeptides present in potent vaccines as potential candidates for use as vaccines.

25. The method of claim 24 which is used to determine the amount of a protein or polypeptide present in potent vaccines.


SEQUENCE LISTING (45 sequences)

SEQ ID NO: 1 - protein, 272 aa, Leptospira interrogans (Canicola)
MKKLSILAISVALFASITACGAFGGLPSLKSSFVLSEDTIPGTNETVKTLLPYGSVINYYGYVKPGQAPDGLVDGNKKAYYLYVWIPAVIAEMGVRMISPTGEIGEPGDGDLVSDAFKAATPEEKSMPHWFDTWIRVERMSAIMPDQIAKAAKAKPVQKLDDDDDGDDTYKEERHNKYNSLTRIKIPNPPKSFDDLKNIDTKKLLVRGLYRISFTTYKPGEVKGSFVASVGLLFPPGIPGVSPLIHSNPEELQKQAIAAEESLKKAASDATK

SEQ ID NO: 2 - protein, 4 aa, Artificial
  Synthetic peptide
MRFA

SEQ ID NO: 3 - protein, 16 aa, Artificial
  Synthetic peptide
AVDQLNEQSSEPNIYR

SEQ ID NO: 4 - protein, 17 aa, Artificial
  Synthetic peptide
ARPQELPFLASIQNQGR

SEQ ID NO: 5 - protein, 20 aa, Artificial
  Synthetic peptide
NYINQYSEVAIQMVMHMQPK

SEQ ID NO: 6 - protein, 17 aa, Artificial
  Synthetic peptide
ISVNNVLPVDFNLMQQK

SEQ ID NO: 7 - protein, 16 aa, Artificial
  Synthetic peptide
VTALYEGFTVQNEANK

SEQ ID NO: 8 - protein, 355 aa, Leptospira interrogans
MRKLSSLISVLVLLMFLGNCAATVDVEYPVFPKDKEGRALQKFLGTIRNVGLAVEAPKKSLWEAIFGEGSSFIDQMPSKVFEAFDKESYYKLTDLSKRADAINEASLSLTGITKNRAKIGNLIGAEAILYIGYQKPYTECSTENKIDAVAAGLKVAGFAASMATGKDVNTGNEPVSKPTGVRMMLIPLDATLIKVETGEVKKAVVSSPAKIFNSVGNLECPSILDSFGQGLDEAAAYIKGRLSPIVKTERIKVFVKDEDEEVKELLQEGYEEIVGETPSFKKAKEAWEKADKKAKGQSWGAKANLATYYFSTGDFEKSIKLYEEAMKLKDADKSYLRELRKRVEATFAVDESNAK

SEQ ID NO: 9 - protein, 281 aa, Leptospira interrogans
MIINHNLAAINSHRVLKFQNNEVAKNMETLSSGMRINRAGDDASGLAVSEKMRTQVKGLRQAERNTEDGMSLIQTTEGYLQETNDIIQRIRVLAIQSSNGIYSAEDRQMIQVEVSQLVDEIDRIASQAEFNKMALLQGDFARGSRTSSMWFHIGPNQHQRERVYIATMTAKSLNLIKADGSLLTLSTAEFANDAIGTLDDALMKINKQRANLGAYFNRLEHASKGLMVAYENIQASESRIRDTDMAEETVAFTKNQILVQSGTAMLAQANVRPQSVLQLLR

SEQ ID NO: 10 - protein, 221 aa, Leptospira interrogans
MRRVFSNLGIWMFFFVLPKIFAAGSQNPSIYTVQKGDTYFSLGKKFKVDYHKIMEWNGKKEKDSLLPGEILKIQKPTSTENQKIVSKNTKSNFVKSESVESSKPNFKFPLKNRTPIQSHFTKLSFAPHKGILFKATRHAEVRPASPGKVLVVDEMEGYKKYVILEHKNGYSTVYANLKTVSVNEGETVDPSKILGSLESGKGLYFQLNHGSSAIDPSLQIR

SEQ ID NO: 11 - protein, 157 aa, Leptospira interrogans
MKFRSIVILMFAVFFTSGALFAEKSTEEHIKSLSSGSDSEKYESAVALGKNKEKSAIPELIQLLNRNNEPKIATAAAISLGKIAEPGDSTIALKNKIISSENGDIVYASLASLLNITTKNEKLEDSTKEAFEYADKNRRSDEFVADLLDLIKKKLKL

SEQ ID NO: 12 - protein, 95 aa, Leptospira interrogans
MIAMLLVTLLRCFSIQEHGVETNNWGHPYIGTQISITSFPCLVELSSILFYIPVPFVILNVPLSFIVDTILLSGDLVIKPERKRPTMDNYYCDTL

SEQ ID NO: 13 - protein, 18 aa, Artificial Sequence
  Synthetic peptide
SSFVLSEDTIPGTNETVK

SEQ ID NO: 14 - protein, 12 aa, Artificial Sequence
  Synthetic peptide
ISFTTYKPGEVK

SEQ ID NO: 15 - protein, 18 aa, Artificial Sequence
  Synthetic peptide
AYYLYVWIPAVIAEMGVR

SEQ ID NO: 16 - protein, 6 aa, Artificial Sequence
  Synthetic peptide
IPNPPK

SEQ ID NO: 17 - protein, 12 aa, Artificial Sequence
  Synthetic peptide
LDDDDDGDDTYK

SEQ ID NO: 18 - protein, 11 aa, Artificial Sequence
  Synthetic peptide
SMPHWFDTWIR

SEQ ID NO: 19 - protein, 8 aa, Artificial Sequence
  Synthetic peptide
IKIPNPPK

SEQ ID NO: 20 - protein, 22 aa, Artificial Sequence
  Synthetic peptide
MISPTGEIGEPGDGDLVSDAFK

SEQ ID NO: 21 - protein, 11 aa, Artificial Sequence
  Synthetic peptide
MSAIMPDQIAK

SEQ ID NO: 22 - protein, 9 aa, Artificial Sequence
  Synthetic peptide
AAKAKPVQK

SEQ ID NO: 23 - protein, 18 aa, Artificial Sequence
  Synthetic peptide
AATPEEKSMPHWFDTWIR

SEQ ID NO: 24 - protein, 18 aa, Artificial Sequence
  Synthetic peptide
AKPVQKLDDDDDGDDTYK

SEQ ID NO: 25 - protein, 6 aa, Artificial Sequence
  Synthetic peptide
EERHNK

SEQ ID NO: 26 - protein, 31 aa, Artificial Sequence
  Synthetic peptide
GSFVASVGLLFPPGIPGVSPLIHSNPEELQK

SEQ ID NO: 27 - protein, 12 aa, Artificial Sequence
  Synthetic peptide
IPNPPKSFDDLK

SEQ ID NO: 28 - protein, 19 aa, Artificial Sequence
  Synthetic peptide
KAYYLYVWIPAVIAEMGVR

SEQ ID NO: 29 - protein, 28 aa, Artificial Sequence
  synthetic peptide
KLSILAISVALFASITACGAFGGLPSLK

SEQ ID NO: 30 - protein, 15 aa, Artificial Sequence
  Synthetic peptide
LDDDDDGDDTYKEER

SEQ ID NO: 31 - protein, 8 aa, Artificial Sequence
  Synthetic peptide
LLVRGLYR

SEQ ID NO: 32 - protein, 27 aa, Artificial Sequence
  Synthetic peptide
LSILAISVALFASITACGAFGGLPSLK

SEQ ID NO: 33 - protein, 10 aa, Artificial Sequence
  Synthetic peptide
QAIAAEESLK

SEQ ID NO: 34 - protein, 11 aa, Artificial Sequence
  Synthetic peptide
QAIAAEESLKK

SEQ ID NO: 35 - protein, 11 aa, Artificial Sequence
  Synthetic peptide
SFDDLKNIDTK

SEQ ID NO: 36 - protein, 14 aa, Artificial Sequence
  Synthetic peptide
SMPHWFDTWIRVER

SEQ ID NO: 37 - protein, 29 aa, Artificial Sequence
  Synthetic peptide
TLLPYGSVINYYGYVKPGQAPDGLVDGNK

SEQ ID NO: 38 - protein, 30 aa, Artificial Sequence
  Synthetic peptide
TLLPYGSVINYYGYVKPGQAPDGLVDGNKK

SEQ ID NO: 39 - protein, 8 aa, Artificial Sequence
  Synthetic peptide
YNSLTRIK

SEQ ID NO: 40 - protein, 16 aa, Artificial Sequence
  Synthetic peptide
GLYRISFTTYKPGEVK

SEQ ID NO: 41 - protein, 9 aa, Artificial Sequence
  Synthetic peptide
HNKYNSLTR

SEQ ID NO: 42 - protein, 29 aa, Artificial Sequence
  Synthetic peptide
MISPTGEIGEPGDGDLVSDAFKAATPEEK

SEQ ID NO: 43 - protein, 14 aa, Artificial Sequence
  Synthetic peptide
MSAIMPDQIAKAAK

SEQ ID NO: 44 - protein, 14 aa, Artificial Sequence
  Synthetic peptide
VERMSAIMPDQIAK

SEQ ID NO: 45 - DNA, 54 bp, Artificial Sequence
  Synthetic DNA
agctcttttgttctgagcgaggacacaatcccagggacaaacgaaaccgtaaaa